(19) United States
(12) Patent Application Publication
(10) Pub. No.: US 2004/0176769 A1
(43) Pub. Date: Sep. 9, 2004

(54) Apparatus and method for expanding bone

(76) Inventors: Masahiro Furutani, Kobe-shi (JP); Takafumi Tsurui, Kobe-shi (JP)

Correspondence Address: Ronald E. Greigg, Unit One Station Square, 1423 Powhatan Street, Alexandria, VA, 22314, US

(21) Appl. No.: 10/794,059

(22) Filed: Mar. 8, 2004

(30) Foreign Application Priority Data
Mar. 7, 2003 (JP) 2003-61166
Oct. 28, 2003 (JP) 2003-366866

(51) Int. Cl. A61B 17/58

(52) U.S. Cl. 606/72

(57) ABSTRACT

The present invention provides an apparatus for expanding bones that is compact in size and is capable of moving the cut-off piece of bone accurately in a desired direction, expanding a bone in a narrow region, minimizing the obstruction to the supply of nutrient to the cut-off piece of bone during the expansion of the bone and other portion, reducing the load on the bone and preventing damage in the oral cavity from occurring during the expansion of the bone. The bone expanding apparatus holds the bone base A1 and the cut-off piece of bone A2 at a small separation gap S and gradually increases the separation gap S thereby to cause new bone to grow in the separation gap S, thereby to fill the separation gap S with the newly generated bone and expand the bone, and comprises the support pillar 10 to be erected and fixed in the bone base A1, the adjust screw 20 that fits onto the support pillar 10 from the outside thereof with the support pillar serving as a center axis of rotation and has thread formed on the circumference, the screw receiving portion 41 that mates with the adjust screw 20 being screwed therethrough, and the mounting fixture 40 having the mount base 42 to be connected and fixed onto the cut-off piece of bone A2.

### BACKGROUND OF THE INVENTION

1. Field of the Invention

The present invention relates to a bone expanding apparatus and a bone expanding method used in expanding bones such as increasing the thickness of alveolar bone, jaw bone or other bone or extending a bone in a certain direction.

2. Description of Related Art

It becomes necessary to expand a bone, in the case of an alveolar bone for example, prior to an operation of planting a root of tooth, called the implant, in the alveolar bone. When a root of tooth is planted in the alveolar bone, planting cannot be carried out properly when the thickness of the alveolar bone is not enough at a position where the root of tooth is to be planted. Therefore, it becomes necessary to increase the thickness of the alveolar bone at a position where the root of tooth is to be planted before planting.

In the case of jaw bone, too, it often becomes necessary to extend a part of the bone outwardly or increase the thickness thereof, in relation to the implant or reconstructing operation.

Now reference will be made to FIG. 13(A), (B), and a prior art example of the bone expanding apparatus of this type will be described below.

The bone expanding apparatus of the prior art substantially comprises a pad 1 and an adjust screw 2. The apparatus is intended to be planted in an alveolar bone, in this example, and lower alveolar bone A among a pair of upper and lower alveolar bones in the case of the example shown in FIG. 13. A portion of the alveolar bone A where it is desired to increase the thickness is cut off so as to separate a cut-off piece of bone A2. The cut-off piece of bone A2 is disposed with a small separation gap S being kept from alveolar bone base A1, with cut surfaces on both sides kept across the small separation gap S.

The cut-off piece of bone A2 is connected to the alveolar bone base A1 and held in position by the pad 1 using a screw 1a or the like. The adjust screw 2 is screwed into a screw receiving portion 1b of the pad 1 so that distal end of the adjust screw penetrates through the cut-off piece of bone A2 and makes contact with the alveolar bone base A1 that opposes thereto. Under this condition, the cut-off piece of bone A2 can be moved so as to gradually increase the separation gap S by turning the adjust screw 2 little by little. As new bone is regenerated in the small separation gap S so as to fill the separation gap S with the new bone, the bone thickness can be increased from T1 to T2 as indicated by a dashed line by increasing the separation gap S along with the regeneration of bone. [Patent Document 1] Japanese Unexamined Patent Publication No. 2003-135478 [Patent Document 2] Japanese Unexamined Patent Publication No. 2003-144460

In the case of the bone expanding apparatus of the prior art, however, due to the fact that the pad 1 must be attached so as to stretch over the cut-off piece of bone A2 and the alveolar bone base A1 and that the cut-off piece of bone A2 must be held in a stable condition, the pad 1 cannot be made too small in area. As a result, there arises such a problem that the portion of bone covered by the pad 1 having a significant area does not receive sufficient supply of nutrient. Also because the pad 1 has a significant area, there is a limitation to decreasing the size of region of the bone to be expanded. Therefore, it has been difficult to expand a bone in a small region for a small number of roots of teeth.

While the cut-off piece of bone A2 is attached to the pad 1, the pad 1 is attached also to the alveolar bone base A1. As a result, a low degree of freedom is allowed for the direction of moving the cut-off piece of bone A2, which can be moved only in one direction that is at right angles to the pad 1.

Also because there is no member for guiding the movement of the cut-off piece of bone A2, the cut-off piece of bone A2 cannot be moved in an accurately controlled direction.

In case the pad 1 is attached to the alveolar bone base A1 in a loose manner in order to make the movement of the cut-off piece of bone A2 easier, position of the cut-off piece of bone A2 becomes unstable. In case the pad 1 is attached to the alveolar bone base A1 in a tight manner, on the other hand, a significant bend resistance of the pad 1 must be overcome to move the cut-off piece of bone A2 and expand the separation gap S by means of the adjust screw 2. This leaves the alveolar bone base A1 under a significant load applied by the adjust screw 2 of which distal end makes contact therewith.

There is also such a problem that the alveolar bone base A1 is subjected to a significant stress applied thereto since the adjust screw 2 makes direct contact with the alveolar bone base A1 at the distal end thereof.

### BRIEF DESCRIPTION OF THE DRAWINGS

FIG. 1 is a front view showing the installation of the bone expanding apparatus according to the first embodiment of the present invention;

FIG. 2 is a longitudinal sectional view showing the installation of the bone expanding apparatus according to the first embodiment of the present invention;

FIG. 3 is an exploded view showing component parts of the bone expanding apparatus according to the first embodiment of the present invention;

FIG. 4 shows the support pillar with (A) in plan view and (B) in front view;

FIG. 5 shows the adjust screw with (A) in plan view and (B) in front view with a part cut away in the longitudinal direction;

FIG. 6 shows the mounting fixture with (A) in plan view and (B) in longitudinal sectional view;

FIG. 7 shows the procedure for expanding bone according to the first embodiment of the present invention;

FIG. 8 is a plan view showing the installation of the bone expanding apparatus according to the second embodiment of the present invention with a part being cut away;

FIG. 9 is a longitudinal sectional view showing the installation of the bone expanding apparatus according to the second embodiment of the present invention;

FIG. 10 is an exploded view showing component parts of the bone expanding apparatus according to the second embodiment of the present invention;

FIG. 11 shows the mounting fixture of the bone expanding apparatus according to the second embodiment of the present invention, with (A) in plan view and (B) in front view;

FIG. 12 shows the procedure for expanding bone according to the second embodiment of the present invention; and

FIG. 13 shows an example of apparatus of the prior art, (A) being a front view showing the apparatus attached to the alveolar bone, and (B) being a longitudinal sectional view thereof.

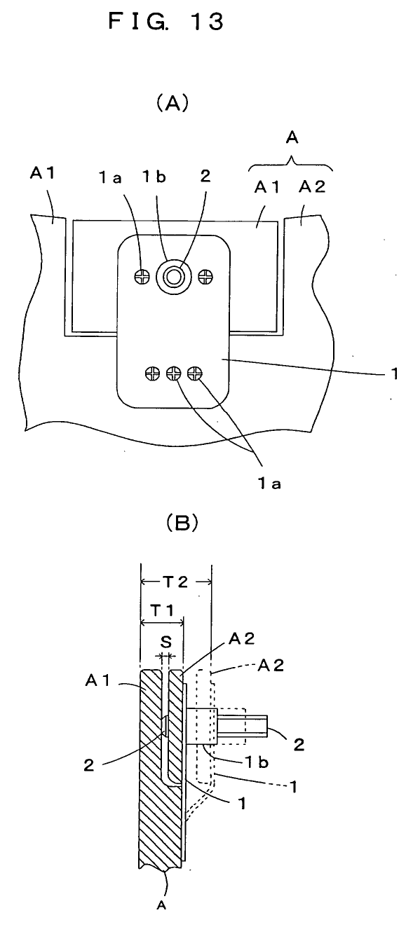

### DESCRIPTION OF REFERENCE NUMERALS

10: Support pillar

11: Round rod portion

12: Threaded portion

13: Joint

14: Stopper portion

20: Adjust screw

21: Inner hole

22: External thread

30: Living tissue protecting cap

40: Mounting fixture

41: Screw receiving portion

41a: Internal thread

42: Mount base

42a: Set screw inserting hole

43: Set screw

50: Alignment assisting pin

51: Large diameter portion

52: Small diameter portion

60: Screw driver

71: First prepared hole

72: Second prepared hole

73: Cutoff line

80: Mounting fixture

81: Screw receiving portion

81a: Internal thread

82: Mount base

82c: Contact surface

83: Set screw

91: Cutoff surface

92: Prepared hole

A: Alveolar bone

A1: Alveolar bone base

A2: Cut-off piece of bone

B: Gingiva

C: Lip flesh

D: Lower jaw bone

D1: Lower jaw bone base

D2: cut-off piece of bone

D21: Side face

E: Mucous membrane of lower jaw bone

P: Predetermine cutoff line

### SUMMARY OF THE INVENTION

Thus the present invention aims at solving the problems of the bone expanding apparatus of the prior art described above, and provides an apparatus for expanding bones that is compact in the overall size and is capable of precisely moving the cut-off piece of bone while maintaining precise position thereof, directing the movement of the cut-off piece of bone precisely in the desired direction, expanding a bone in a narrow region, minimizing the obstruction to the supply of nutrient to the cut-off piece of bone during the expansion of the bone, reducing the load on the bone during the expansion of the bone and, of course, minimizing the possibility of injury caused in the oral cavity during the expansion of the bone.

The bone expanding apparatus according to the present invention that solves the problems described above holds bone base and a cut-off piece of bone, which is a part of the bone base being cut off therefrom, at a small distance from each other and gradually increases the separation gap thereby to cause new bone to grow in the separation gap, thereby to fill the separation gap with the newly generated bone and expand the bone, and comprises a support pillar to be connected and fixed in the bone base, an adjust screw that fits onto the support pillar from the outside thereof with the support pillar serving as a center axis of rotation and has external thread formed on the circumference, a screw receiving portion that mates with the adjust screw which is screwed therethrough, and a mounting fixture having mount bases to be connected and fixed to the cut-off piece of bone, as a first feature.

The bone expanding apparatus according to the present invention, in addition to the first feature described above, has a stopper portion provided at a mid position thereof to make contact with and stop the distal end of the adjust screw, as a second feature.

The bone expanding apparatus according to the present invention, in addition to the first feature described above, has thread formed on the distal end portion of the support pillar so as to be screwed into the bone base and fixed therein, as a third feature.

The bone expanding apparatus according to the present invention, in addition to the third feature described above, has a detachable joint at the head of the support pillar for detachably attaching a screw driver used as an auxiliary tool for screwing the threaded distal end portion into the bone base, as a fourth feature.

The bone expanding apparatus according to the present invention, in addition to the first feature described above, has a living tissue protecting cap detachably attached to the head of the adjust screw, as a fifth feature.

The bone expanding apparatus according to the present invention, in addition to the first feature described above, has the support pillar to be erected and fixed in the bone base and the adjust screw that fits the support pillar from the outside thereof which are erected while penetrating the cut-off piece of bone and the mount bases of the mounting fixture being installed around the screw receiving portion with which the adjust screw mates while penetrating therethrough by screwing, as a sixth feature.

The bone expanding apparatus according to the present invention, in addition to the sixth feature described above, has an alignment assisting pin as a mount assisting member that is temporarily fitted in prepared holes formed in the bone base and in the cut-off piece of bone prior to mounting of the support pillar and the adjust screw when connecting and fixing the mounting fixture onto the cut-off piece of bone, while being fitted into the screw receiving portion of the mounting fixture at the same time, and has an outer diameter that just fits the prepared holes and an outer diameter that just fits the screw receiving portion of the mounting fixture, as a seventh feature.

The bone expanding apparatus according to the present invention, in addition to the first feature described above, has the support pillar to be erected and fixed in the bone base and the adjust screw that fits the support pillar from the outside thereof which are erected at a position sideways to the cut-off piece of bone and the mount bases of the mounting fixture which are connected and fixed to the cut-off piece of bone at a position sideways to the screw receiving portion with which the adjust screw mates while penetrating therethrough by screwing, as an eighth feature.

The bone expanding apparatus according to the present invention, in addition to the first feature described above, is intended for applications of expanding alveolar bones and jaw bones, as a ninth feature.

The bone expanding method according to the present invention is a method of holding the bone base and the cut-off piece of bone, which is a part of the bone base being cut off therefrom, at a small distance from each other and gradually increases the separation gap thereby to cause new bone to grow in the separation gap, thereby to fill the separation gap with the newly generated bone and expand the bone, the method comprising the step of forming the prepared hole in the bone base for erecting the support pillar and forming the prepared hole concentrically therewith in the cut-off piece of bone for passing the support pillar and the adjust screw therethrough, the step of temporarily fitting the alignment assisting pin in the prepared hole and fitting the screw receiving portion of mounting fixture for fixing the cut-off piece of bone to the alignment assisting pin thereby to connect and fix the mount bases of the mounting fixture into the cut-off piece of bone with the screw receiving portion of the mounting fixture being centered, the step of removing the alignment assisting pin from the prepared hole and passing the support pillar through the screw receiving portion of the mounting fixture and through the prepared hole of the cut-off piece of bone and erecting and fixing the support pillar into the prepared hole of the bone base, the step of fitting the adjust screw onto the support pillar that has been erected from the outside thereof with the support pillar serving as a center axis of rotation and screwing the external thread formed on the circumference of the adjust screw into the internal thread of the screw receiving portion, and the step of turning the adjust screw so as to gradually expand the separation gap between the bone base and the cut-off piece of bone, as a tenth feature.

The bone expanding method according to the present invention is a method of holding the bone base and the cut-off piece of bone, which is a part of the bone base being cut off therefrom, at a small distance from each other and gradually increasing the separation gap so as to cause new bone to grow in the separation gap, thereby to fill the separation gap with the newly generated bone and expand the bone, the method comprising the step of forming a cut-off surface for erecting the support pillar by cutting into the bone base at a position adjacent to a predetermined cutting position, the step of forming the prepared hole in the cut-off surface for erecting the support pillar and temporarily erecting the support pillar therein, the step of fitting the mounting fixture for fixing the cut-off piece of bone onto the erected support pillar at the screw receiving portion, putting the mount bases of the mounting fixture onto the side face of the bone to be cut off adjacent to the cut-off surface for erecting the support pillar and drilling a mount hole in the bone to be cut off with the set screw inserting hole of the mounting portion, the step of temporarily removing the support pillar and cutting off the cut-off piece of bone at the predetermined cutting position, the step of erecting the support pillar again in the prepared hole of the bone base, the step of fitting the adjust screw onto the support pillar that has been erected from the outside thereof so as to turn around the support pillar serving as a center axis of rotation, the step of fixing the mount bases of the mounting fixture that has been screwed onto the thread formed on the circumference of the adjust screw into the cut-off piece of bone with set screws, and the step of turning the adjust screw so as to gradually expand the separation gap between the bone base and the cut-off piece of bone, as an eleventh feature.

According to the bone expanding apparatus of claim 1, the mounting fixture having the screw receiving portion that mates with the adjust screw by screwing can be moved back and forth together with the cut-off piece of bone that is connected and fixed to the mounting fixture by turning the adjust screw that fits onto the support pillar, which has been erected in the bone base, from the outside of the support pillar. Specifically, the separation gap between the cut-off piece of bone and the bone base can be gradually increased by turning the adjust screw, for example one full turn once a day or a certain number of turns in the same direction at certain intervals. As the separation gap is increased, new bone is regenerated in the separation gap so as to fill the separation gap. As a result, the bone can be expanded.

Since the support pillar serves as the central axis of rotation for the adjust screw, the mounting fixture and the cut-off piece of bone to which the mounting fixture is fixed are surely guided in the moving direction by the support pillar, so that the cut-off piece of bone can be moved accurately while being guided by the support pillar.

The cut-off piece of bone can also be moved in any desired direction by erecting the support pillar in a desired angle instead of erecting it at right angles to the cut surface of the bone base.

The mounting fixture that is moved by turning the adjust screw is fixed with respect to the cut-off piece of bone, but is not fixed with respect to the bone base or other part. Therefore, the mounting fixture and the cut-off piece of bone can be easily moved by turning the adjust screw without exerting a large force and, since a large force is not exerted, loads on the bone base and on the other portion of bone can be reduced.

Since only the mounting fixture and the cut-off piece of bone are moved and positions of the adjust screw and of the support pillar are not affected by turning the adjust screw, such a trouble does not occur as the adjust screw or the support pillar are caused to protrude further and injure the inside of the oral cavity during the period of bone expansion.

The mounting fixture may be fixed only onto the cut-off piece of bone, and it is not necessary to be fixed over the bone base and the cut-off piece of bone. Consequently, the mounting fixture may be made smaller in area and the obstruction to the supply of nutrient via the periosteum to the bone by the mounting fixture can be minimized.

According to the bone expanding apparatus of claim 2, besides the effects of the constitution of claim 1 described above, such troubles can be surely prevented as the tip of the adjust screw that is being turned makes direct contact with the bone base to cause injury thereon or applying a concentrated load on the bone, since the stopper is provided at a mid position of the support pillar so that the tip of the adjust screw makes contact with the stopper and is received thereby.

According to the bone expanding apparatus of claim 3, besides the effects of the constitution of claim 1 described above, the support pillar can be erected and fixed in the bone base by screwing the threaded portion of the distal end of the support pillar into the bone base, thus the erection in the bone base can be carried out reliably with a simple constitution.

According to the bone expanding apparatus of claim 4, besides the effects of the constitution of claim 3 described above, since a joint is provided for attaching a screw driver detachably to the head of the support pillar, the support pillar can be easily and reliably screwed into the bone base and fixed therein by turning the screw driver as an auxiliary tool.

According to the bone expanding apparatus of claim 5, besides the effects of the constitution of claim 1 described above, injury on living tissues such as mucous membranes that wound be caused by the adjust screw disposed to protrude in the oral cavity can be prevented by attaching the living tissue protecting cap to the head of the adjust screw.

According to the bone expanding apparatus of claim 6, besides the effects of the constitution of claim 1 described above, since the support pillar and the adjust screw are erected so as to penetrate the cut-off piece of bone, the space for erecting the support pillar and the adjust screw can be secured within a region of the cut-off piece of bone so that the support pillar and the adjust screw can be erected in a smaller space. Also because the mount bases of the mounting fixture are provided around the screw receiving portion of the mounting fixture which is located at the center, the cut-off piece of bone can be easily fixed by the mounting fixture, firmly and reliably. It needs not to say that the mounting fixture can be made in a compact construction with the screw receiving portion located at the center thereof. Thus obstruction of the supply of nutrient to the cut-off piece of bone by the mounting fixture can be minimized. Also because the area required for the mounting fixture can be made smaller, the method can be applied to a case of smaller area of cut-off piece of bone and a case of smaller area of bone expansion.

According to the bone expanding apparatus of claim 7, besides the effects of the constitution of claim 6 described above, since the alignment assisting pin is provided that has an outer diameter which just fits the prepared holes that are drilled in the bone base and in the cut-off piece of bone prior to the installation of the support pillar and the adjust screw and fits in the screw receiving portion of the mounting fixture, the screw receiving portion of the mounting fixture can be accurately and easily aligned with the adjust screw when fixing the mounting fixture onto the cut-off piece of bone by using the alignment assisting pin. This makes it possible to accurately mate the adjust screw and the screw receiving portion of the mounting fixture by screwing, when the adjust screw is attached later. Moreover, the mounting fixture and the cut-off piece of bone can be easily and smoothly moved by turning by turning the adjust screw.

According to the bone expanding apparatus of claim 8, in addition to the effects of the constitution of claim 1 described above, since the support pillar erected in the bone base and the adjust screw are erected sideways to the cut-off piece of bone, it is not necessary to drill a through hole in the cut-off piece of bone. As a result, necessity for accurately drilling a thorough hole, space for drilling the thorough hole and necessity for the cut-off piece of bone to have high strength to endure the drilling of the thorough hole are eliminated.

The mounting fixture can be easily connected and fixed to the cut-off piece of bone by means of the mount bases at a position sideways to the screw receiving portion.

According to the bone expanding apparatus of claim 9, besides the effects of the constitution of claim 1 described above, since alveolar bone and jaw bone are set as the object of the expansion, bone expansion including the thickness or length of the alveolar bone or jaw bone can be easily and surely carried out, so as to carry out the implant operation of planting root of tooth in the alveolar bone or other reconstruction operation easily and surely.

Also according to bone expanding method of claim 10, it is made possible to erect the support pillar, fit the adjust screw freely rotatably onto the support pillar from the outside thereof, mating the mounting fixture with the adjust screw by screwing and connecting and fixing the mounting fixture to the cut-off piece of bone, thereby to hold the bone base and the cut-off piece of bone located near to each other. Then the cut-off piece of bone can be moved so as to gradually increase the separation gap between the cut-off piece of bone and the bone base by turning the adjust screw.

When fixing the mounting fixture to the cut-off piece of bone, the mount bases of the mounting fixture can be easily and surely connected and fixed to the cut-off piece of bone under such a condition as the center of the screw receiving portion of the mounting fixture is aligned with the support pillar and the adjust screw, by temporarily fitting the alignment assisting pin into the prepared holes of the bone base and the cut-off piece of bone that have been drilled in advance for erecting the support pillar and the adjust screw, and fitting the screw receiving portion of the mounting fixture with the alignment assisting pin. With this constitution, when the support pillar is erected and the adjust screw is attached thereto later, it is made possible to mate the mounting fixture that is fixed onto the cut-off piece of bone and the adjust screw in alignment with each other by screwing, thereby moving the mounting fixture and the cut-off piece of bone that is fixed thereto accurately and smoothly.

According to bone expanding method of claim 11, the support pillar is erected at a position adjacent to the cut-off piece of bone, the adjust screw is fitted freely rotatably onto the support pillar from the outside thereof, the mounting fixture is mated with the adjust screw by screwing and the mounting fixture is connected and fixed to the adjacent cut-off piece of bone, thereby it is made possible to hold the bone base and the cut-off piece of bone located near to each other. Then the cut-off piece of bone can be moved so as to gradually increase the separation gap between the cut-off piece of bone and the bone base by turning the adjust screw.

With regard to fixing of the mounting fixture to the cut-off piece of bone, it is made possible to temporarily erect the support pillar in the prepared hole that has been drilled in the cut-off surface for erecting the support pillar formed at a position adjacent to the predetermined cutting position, fit the mounting fixture onto the temporarily erected support pillar at the screw receiving portion, put the mount bases of the mounting fixture to the side face of the bone to be cut off so as to mate the surfaces thereof while at the same time drilling a mounting hole that corresponds to the bone to be cut off, thereby to erect the support pillar and fit the adjust screw to the support pillar from the outside thereof after cutting off the cut-off piece of bone and, under the condition that the mounting fixture is screwed onto the adjust screw, connect the mount bases of the mounting fixture to the cut-off piece of bone in alignment with the adjust screw easily by using the set screw.

### DESCRIPTION OF PREFERRED EMBODIMENTS

Preferred embodiments of the bone expanding apparatus and method of the present invention will now be described with reference to the following accompanying drawings.

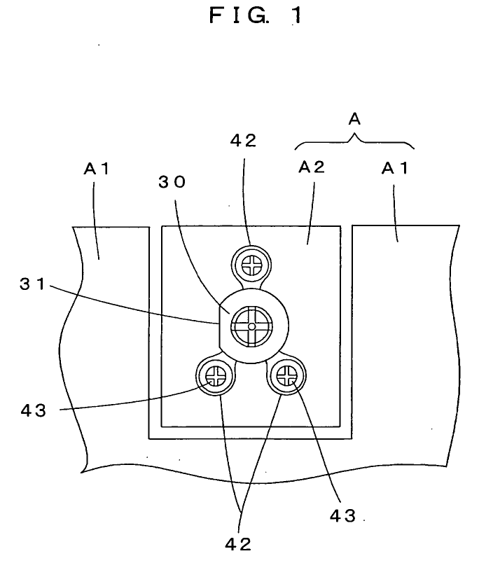

FIG. 1 is a front view showing the installation of the bone expanding apparatus according to the first embodiment of the present invention,

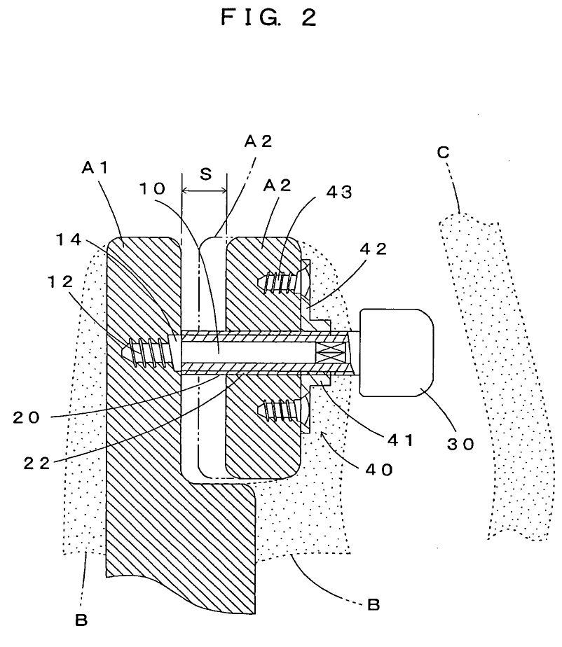

FIG. 2 is a longitudinal sectional view thereof,

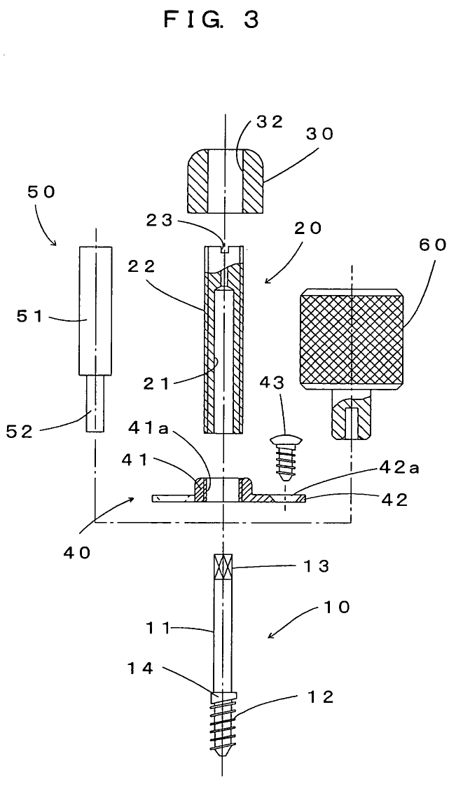

FIG. 3 is an exploded view showing component parts of the bone expanding apparatus according to the first embodiment, and

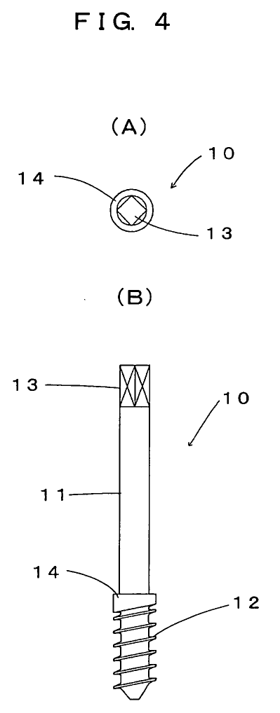

FIG. 4 shows the support pillar with (A) in plan view and (B) in front view.

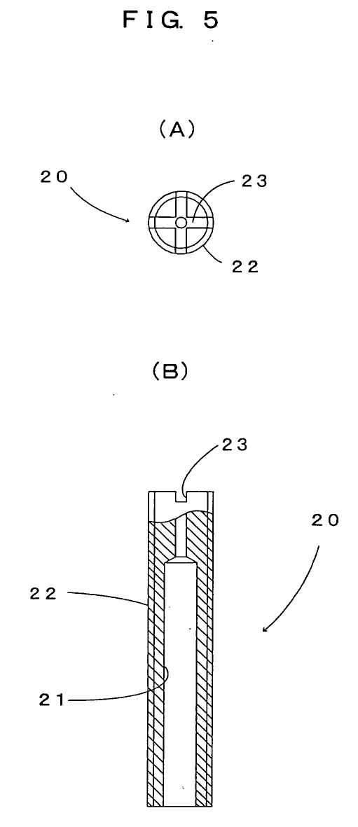

FIG. 5 shows the adjust screw with (A) in plan view and (B) in front view with a part cut away in the longitudinal direction.

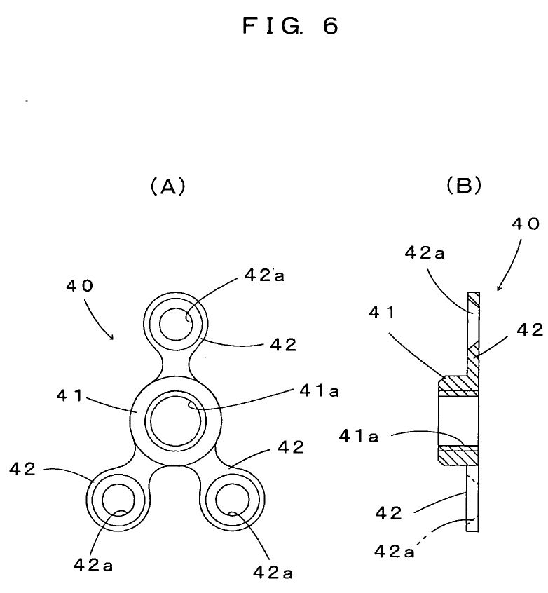

FIG. 6 shows the mounting fixture with (A) in plan view and (B) in longitudinal sectional view.

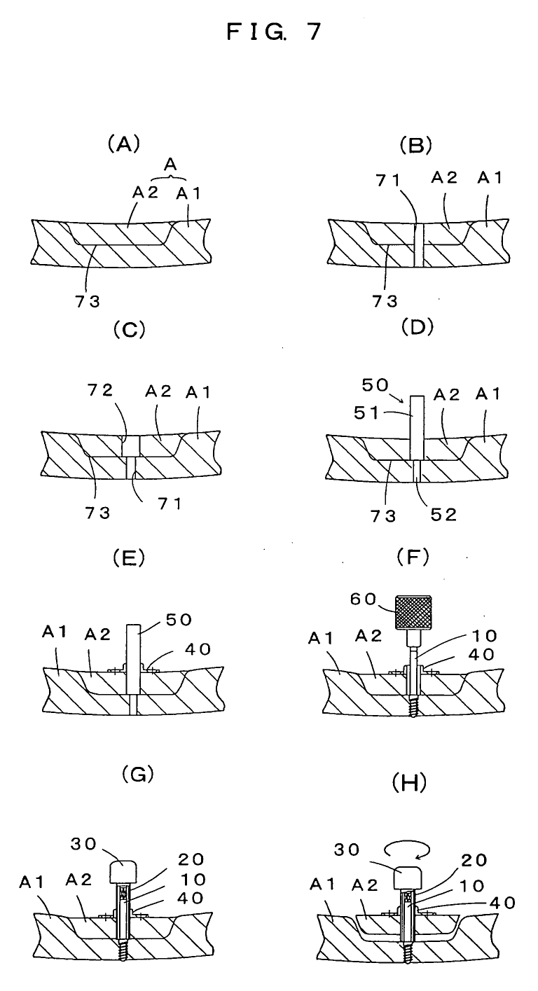

FIG. 7(A) through (H) show the procedure for expanding a bone.

Reference will be made first to FIG. 1 and FIG. 2 where application of the bone expanding apparatus according to the first embodiment to alveolar bone is shown, more specifically application of the bone expanding apparatus according to the first embodiment to lower alveolar bone is shown. That is, a part of the lower alveolar bone is cut off to make a cut-off piece of bone A2, the bone expanding apparatus is installed so as to hold the cut-off piece of bone A2 in proximity of the alveolar bone base A1, and the separation gap S is gradually increased.

In FIG. 2, letter B denotes gingival and C denotes lip flesh.

Now making reference also to FIG. 3, the bone expanding apparatus according to the first embodiment comprises a support pillar 10, an adjust screw 20 that fits the support pillar 10 freely rotatably from the outside thereof, a living tissue protecting cap 30 that is attached to the adjust screw 20, a mounting fixture 40 that is fixed onto the cut-off piece of bone A2, an alignment assisting pin 50 used as an assisting tool when installing the apparatus, and a screw driver 60 used when installing the apparatus.

Now making reference also to FIG. 4, the support pillar 10 passes through the cut-off piece of bone A2 so as to be erected and fixed as the distal end thereof invades into the alveolar bone base A1. While the body of the support pillar 10 is a round rod portion 11, the distal end thereof is a threaded portion 12, and the head makes a joint 13 of prism shape. The support pillar 10 has, at a mid portion thereof and at the top of the threaded portion 12, a stepped portion having a diameter a little larger than that of the round rod portion 11, so as to provide a stopper portion 14.

The round rod portion 11 receives the adjust screw 20 fitted thereon freely rotatably from the outside.

The threaded portion 12 is screwed into the alveolar bone base A1 so that the support pillar 10 is erected and fixed in the alveolar bone base A1.

The joint 13 engages with the screw driver 60 freely detachably so that the threaded portion 12 can be screwed into the alveolar bone base A1 thereby to fix the support pillar 10 in the alveolar bone base A1 by turning the screw driver 60.

The stopper portion 14 receives, on the top surface thereof, the tip (lower end) of the adjust screw 20 by making contact therewith.

Now with reference made also to FIG. 5, the adjust screw 20 fits onto the support pillar 10 freely rotatably from the outside, and mates with the mounting fixture 40 by screwing on the outside thereof. The adjust screw 20 has an inner hole 21 that fits the support pillar 10 freely rotatably from the outside thereof, and external thread 22 that mates with the mounting fixture 40 by screwing on the outside thereof, with the head fitted with the living tissue protecting cap 30 attached thereto detachably. The living tissue protecting cap 30 may also be integrally attached to the head of the adjust screw 20.

Size of the inner hole 21 for fitting with the outer diameter of the round rod portion 11 is determined so that the adjust screw can rotate without play around the support pillar 10 that serves as the axis of rotation, when the inner hole 21 is fitted onto the support pillar 10 from the head to the round rod portion 11 on the outside thereof. Length of the inner hole 21 is set so as to be capable of receiving the portion of the support pillar 10 above the stopper 14 into the inner hole 21.

The external thread 22 is formed at the same pitch as that of the internal thread 42 of the mounting fixture 40 in the screw receiving portion 41 thereof which will be described later.

The adjust screw 20 has a slot 23 formed on the top end face thereof, so that the adjust screw 20 can be rotated around the support pillar 10 serving as the axis of rotation by engaging a screw driver or the like in the slot 23 and turning.

The living tissue protecting cap 30 is attached to the head of the adjust screw 20 detachably, but may also be attached integrally as described above. The living tissue protecting cap 30, that is made of silicone, for example, plays the roles of protecting the mucous membranes or the like on the inside of the lip flesh C in the oral cavity from injury (refer to FIG. 2). Without the living tissue protecting cap 30, tip of the adjust screw 20 can easily injure the lip flesh C.

The living tissue protecting cap 30 is formed so as to be penetrated by a through hole 32 at the center thereof so that a screw driver or the like can be inserted to turn the adjust screw 20 for adjustment.

The living tissue protecting cap 30 has a flat face 31 in part thereof (refer to FIG. 1) so that the angular position of the adjust screw 20 can be known by the flat face 31.

Now with reference made also to FIG. 6, the mounting fixture 40 comprises a screw receiving portion 41 having internal thread 41a formed thereon and mount bases 42 having a plurality (3 in the embodiment) of feet formed radially around the screw receiving portion 41, with set screw inserting holes 42a formed in each foot of the mount bases 42.

The internal thread 41a of the screw receiving portion 41 is formed at the same pitch as that of the external thread 22 of the adjust screw 20, as described previously.

The mounting fixture 40 can be fixed onto the cut-off piece of bone A2 by putting the mount base 42 to the cut-off piece of bone A2 and screwing the set screw 43 through the set screw inserting hole 42a into the cut-off piece of bone A2.

A prepared hole having a slightly smaller diameter may be drilled in preparation for screwing the set screw 43 into the cut-off piece of bone A2.

The alignment assisting pin 50 is an auxiliary tool used to accurately aligning the screw receiving portion 41 of the mounting fixture 40 with the support pillar 10 when fixing the mounting fixture 40 onto the cut-off piece of bone A2.

The alignment assisting pin 50 is a round rod having a step at a mid point thereof, with the portion above the step being a large diameter portion 51 and the portion below the step being a small diameter portion 52.

The large diameter portion 51 has such an outer diameter that just fits in a second prepared hole 72 drilled in the cut-off piece of bone A2 that will be described later and just fits in the screw receiving portion of the mounting fixture 40. To just fit means that the large diameter portion 51 of the alignment assisting in 50 fits in the second prepared hole 72 snugly without play, and the large diameter portion 51 of the alignment assisting in 50 penetrates the screw receiving portion 41 of the mounting fixture 40 and can rotate but never plays.

The small diameter portion 52 of the alignment assisting pin 50 has such an outer diameter that just fits in a first prepared hole 71 drilled in the alveolar bone base A1 that will be described later. To just fit means that the small diameter portion 52 of the alignment assisting pin 50 fits in the first prepared hole 71 snugly without play.

The screw driver 60 is an auxiliary tool used for screwing and fixing the support pillar 10 into the alveolar bone base A1. The screw driver 60 can be attached freely detachably to the joint 13 provided at the head of the support pillar 10, to be used only when screwing and fixing the support pillar 10 into alveolar bone base A1, and is removed thereafter.

The screw driver 60 may not be necessarily as shown in the drawing, and may have any configuration that can screw the support pillar 10.

With reference made to FIG. 7, the process of installing the apparatus for expanding the alveolar bone and the method of expanding the bone will be described below. FIG. 7(A) through (H) are sectional views of the alveolar bone A in the horizontal plane. Top of the drawing corresponds to the front side of the bone.

First, the alveolar bone A is cut at a predetermined position to make the cut-off piece of bone A2 (A). The cut-off piece of bone A2 is not completely cut off, and remains partially connected to the alveolar bone base A1.

Then the first prepared hole 71 is drilled in horizontal direction in the alveolar bone base A (B). Inner diameter of the first prepared hole 71 is slightly smaller than that of the threaded portion 12 of the support pillar 10. Depth of the first prepared hole 71 in the alveolar bone base A1 may be such that can firmly hold the support pillar 10 erected therein by screwing the threaded portion 12, and it is not necessary to penetrate through the alveolar bone base A1.

Then the second prepared hole 72 is drilled in the alveolar bone A concentrically with the first prepared hole 71 (C). The second prepared hole 72 is drilled to a depth equal to the depth of the cut-off line of the cut-off piece of bone A2, namely such as just penetrates the cut-off piece of bone A2. Inner diameter of the second prepared hole 72 is such that the large diameter portion 51 of the alignment assisting pin 50 just fits therein. This diameter is also such that the adjust screw 20 just fits therein.

Then the alignment assisting pin 50 is inserted into the prepared holes 71, 72 (D), the mounting fixture 40 is fitted to the large diameter portion 51 of the alignment assisting pin 50 at the screw receiving portion 41, the mount base 42 of the mounting fixture 40 is put into contact with the cut-off piece of bone A2 and, under this condition, the mounting fixture 40 is fixed onto the cut-off piece of bone A2 by using the set screw 43 (E).

Fixation is done by screwing the set screw 43 through the set screw inserting hole 42a of the mount base 42 of the mounting fixture 40 into the cut-off piece of bone A2. This causes the mounting fixture 40 to be positioned concentrically around the prepared holes 71, 72.

Then the cut-off piece of bone A2 is completely separated from the alveolar bone base A1, and the alignment assisting pin 50 is removed from the prepared holes 71, 72. Reference numeral 73 denotes the cut-off line.

Then the support pillar 10 is inserted into prepared holes 71, 72, and the support pillar 10 is turned by using the screw driver 60 so as to screw the threaded portion 12 into the prepared hole 71 of the alveolar bone base A1, so as to be erected and fixed therein (F).

Depth of screwing is such that the stopper 14 of the support pillar 10 becomes flush with the alveolar bone base A1 or the stopper 14 comes out a little therefrom.

When the erection of the support pillar 10 has been completed, the screw driver 60 is removed and the adjust screw 20 having the living tissue protecting cap 30 attached thereto is fitted onto the support pillar 10 and is screwed into the screw receiving portion 41 of the mounting fixture 40, and I screwed in till the distal end of the adjust screw 20 makes contact with the stopper 14 of the support pillar 10 (G). The living tissue protecting cap 30 may also be attached to the adjust screw 20 later.

The above operations complete the installation of the apparatus.

When installation is complete, the adjust screw 20 is turned so as to hold the alveolar bone base A1 and the cut-off piece of bone A2 at a small distance from each other (H).

Thereafter, the adjust screw 20 is turned at certain time intervals so as to gradually increase the distance between the alveolar bone base A1 and the cut-off piece of bone A2, so that the space between the alveolar bone base A1 and the cut-off piece of bone A2 is filled with newly generated bone while the bone grows.

In the process described above, the support pillar 10 is erected at right angles to the alveolar bone base A1 in basic practice, although it may be erected at a certain angle from the perpendicular direction. Depending on the condition of the bone, position of the cut-off piece of bone A2 and the direction of increasing the thickness of the bone, it may be better to move the cut-off piece of bone A2 in a direction inclined from the direction perpendicular to the alveolar bone base A1. According to the present invention, since the cut-off piece of bone A2 is guided by the support pillar 10 to move, the cut-off piece of bone A2 can be moved in a desired direction by adjusting the direction of erecting the support pillar 10. Thus the direction of increasing the bone thickness can also be set in the desired direction.

The separation gap S between the alveolar bone base A1 and the cut-off piece of bone A2 is set to a small distance when the apparatus is first installed. The initial value of the separation gap S is set such that new bone can grow easily in the space S. When installation of the bone expanding apparatus is completed, the adjust screw 20 is turned so as to set the separation gap between the cut-off piece of bone A2 and the alveolar bone base A1 to the initial distance, for example 0.5 mm.

When the external thread 22 of the adjust screw 20 is formed with a pitch of, for example, 0.5 mm, the cut-off piece of bone A2 is moved 0.5 mm away from the alveolar bone base A1 by turning the adjust screw 20 by one full turn with a screw driver. Thus the separation gap S between the cut-off piece of bone A2 and the alveolar bone base A1 can be set to the initial distance of 0.5 mm by making one turn of the adjust screw from the state of the cut-off piece of bone A2 making contact with the alveolar bone base A1.

After setting the initial distance, the separation gap S between the cut-off piece of bone A2 and the alveolar bone base A1 is gradually increased over time (H), for example, by making one turn of the adjust screw 20 once a day. This increases the separation gap S by 0.5 mm a day, while the separation gap S is filled with newly generated bone.

When the separation gap has been expanded to a predetermined distance, the apparatus is removed from the alveolar bone base A1 and the cut-off piece of bone A2. This completes the series of operations for expanding the bone.

Since the tip of the adjust screw 20 is stopped by the stopper 14 of the support pillar 10, turning the adjust screw 20 does not cause the adjust screw 20 to make direct contact with the alveolar bone base A1. Thus no load is applied to the alveolar bone base A1, and any adverse effects of such a load can be prevented.

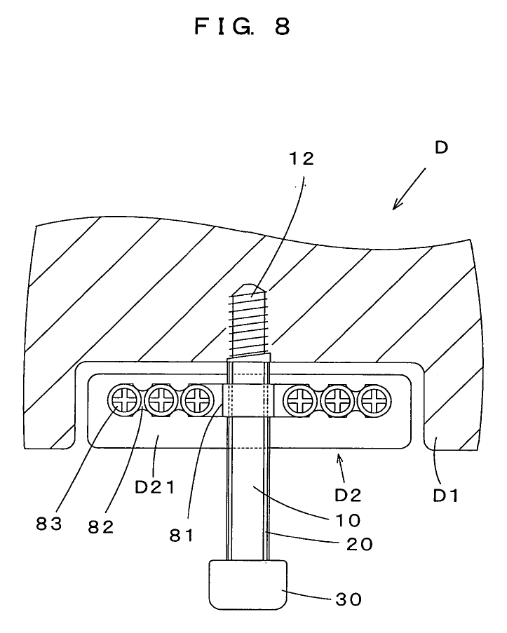

Now the bone expanding apparatus according to second embodiment of the present invention will be described below by making reference to FIG. 8 through FIG. 12.

FIG. 8 is a plan view showing the installation of the bone expanding apparatus according to the second embodiment with a part being cut away,

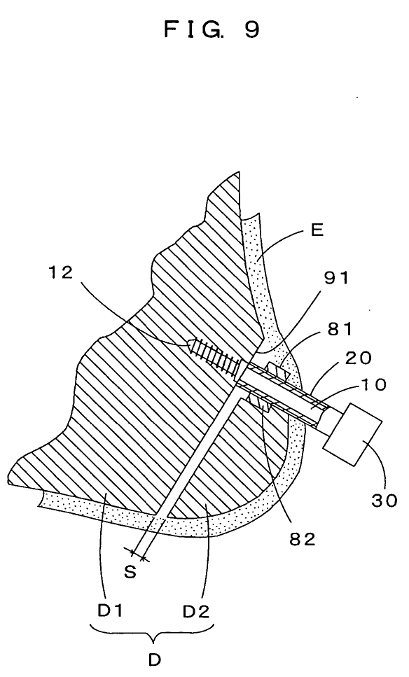

FIG. 9 is a longitudinal sectional view thereof, and

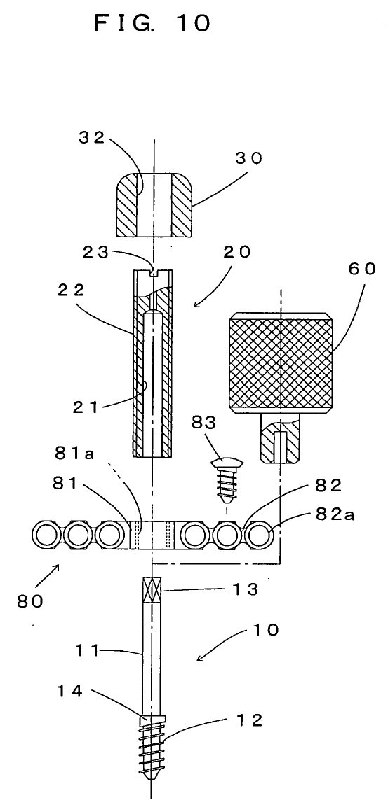

FIG. 10 is an exploded view showing component parts of the bone expanding apparatus according to the second embodiment.

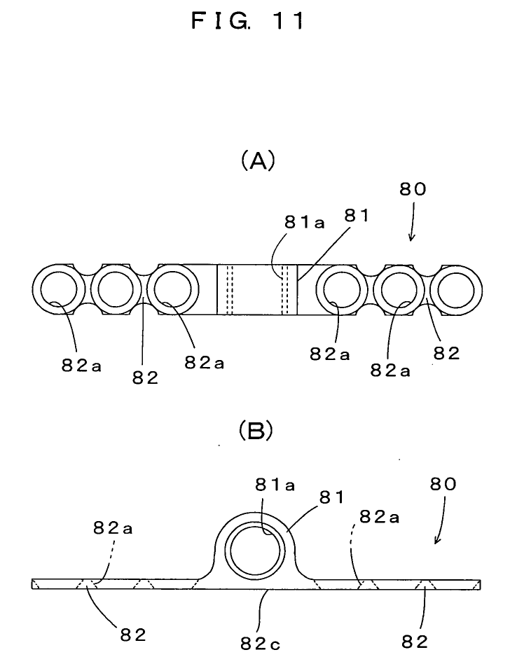

FIG. 11 shows a mounting fixture (A) in plan view and (B) in front view.

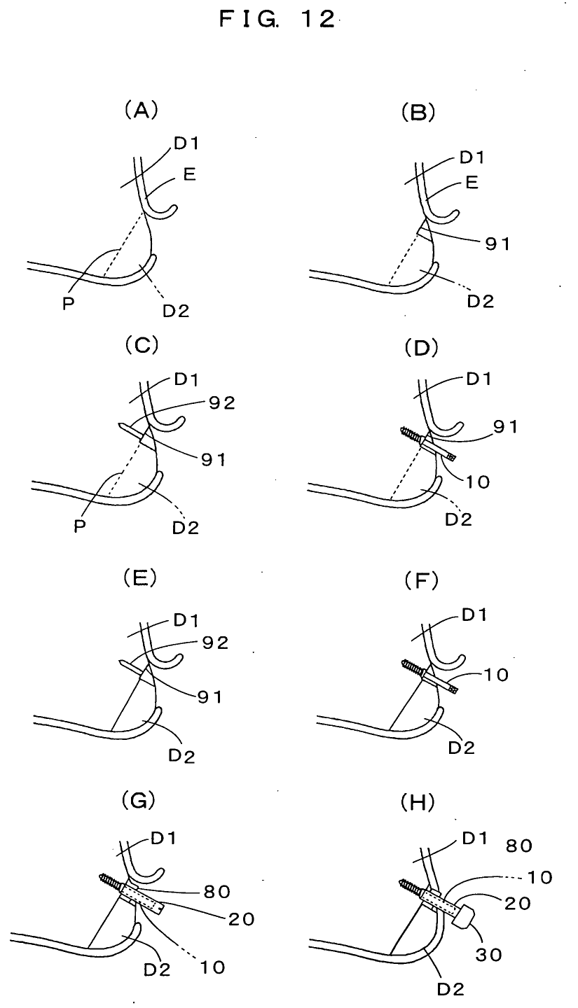

FIG. 12(A) through (H) show the procedure for expanding a bone.

Reference will be made first to FIG. 8 and FIG. 9 where application of the bone expanding apparatus according to the second embodiment to the extension of a jaw bone is shown. That is, a part of the lower jaw bone D is cut off to make a cut-off piece of bone D2, the bone expanding apparatus is installed so as to hold the cut-off piece of bone D2 in proximity of the lower jaw bone base D1, and the separation gap S is gradually increased.

In FIG. 9, E denotes mucous membrane of the lower jaw bone D.

Now with reference made also to FIG. 10, the bone expanding apparatus according to the second embodiment comprises the support pillar 10, the adjust screw 20 that fits the support pillar 10 freely rotatably from the outside thereof, the living tissue protecting cap 30 that is attached to the adjust screw 20, a mounting fixture 80 that is fixed onto the cut-off piece of bone D2, and the screw driver 60 used when installing the apparatus.

The support pillar 10, the adjust screw 20, the living tissue protecting cap 30 and the screw driver 60 have substantially the same functions and shapes as those of the first embodiment, and will therefore be assigned with the same reference numerals and description thereof will be omitted.

In the second embodiment, the mounting fixture 80 is different from the mounting fixture 40 of the first embodiment. The alignment assisting rod 50 of the first embodiment is not used in the second embodiment.

Now with reference made also to FIG. 11, the mounting fixture 80 comprises a screw receiving portion 81 having internal thread 81a formed thereon and a mount base 82 having set screw inserting holes 82a formed in the mount base 82.

The mount base 82, unlike the mount bases 42 of the mounting fixture 40 of the first embodiment, is not installed around the screw receiving portion 81 located at the center but is constituted so as to be connected to the cut-off piece of bone D2 at a position sideways to the screw receiving portion 81. Specifically, the mount base 82 is formed to extend sideways from the screw receiving portion 81 and is constituted so that a contact surface 82C of the screw receiving portion 81 that makes contact with the cut-off piece of bone D2 becomes substantially parallel to the center axis of the screw receiving portion 81. The mount base 82 has a set screw inserting hole 82a drilled in a direction substantially at right angles to the center axis of the screw receiving portion 81 (the center axis of the internal thread 81a).

The mounting fixture 80 comprising the screw receiving portion 81 and the mount base 82 disposed in such an arrangement allows it to connect the mounting fixture 80 to the cut-off piece of bone D2 without passing the support pillar 10 or the adjust screw 20 through the cut-off piece of bone D2. In this case, since it is not necessary to drill a hole in the cut-off piece of bone D2 for passing the support pillar 10 or the adjust screw 20 therethrough, there is no need to give consideration to the strength in conjunction with the formation of through hole in the cut-off piece of bone D2 and no need to secure an extra space for drilling the through hole, and therefore smaller cut-off piece of bone D2 can be extended.

The internal thread 81a of the screw receiving portion 81 is constituted so as to mesh with the external thread 22 of the adjust screw 20 with the same pitch.

Thus it is made possible to put the mounting fixture 82 into contact with the cut-off piece of bone D2 via the contact surface 82C and screw the set screw 83 through the set screw inserting hole 82a into the cut-off piece of bone D2, thereby to fix the mounting fixture 80 onto the cut-off piece of bone D2.

The fixation of the mounting fixture 80 onto the cut-off piece of bone D2 is made on the side face D21 of the cut-off piece of bone D2.

A prepared hole of a smaller diameter is drilled in the cut-off piece of bone D2 in advance, for screwing the set screw 83 into the cut-off piece of bone D2.

Now with reference made to FIG. 12, the method of installing the apparatus for expanding the lower jaw bone and the method of expanding the bone will be described below. FIG. 12(A) through (H) are longitudinal sectional views of the lower jaw bone D.

First, mucous membrane E and periosteum are cut at the predetermined cutting position for extending the lower jaw bone D so as to expose the lower jaw bone D. Then the predetermined cutting position of the cut-off piece of bone D2 (predetermined line of cutoff P is indicated by dashed line) and the direction of extending the cut-off piece of bone D2 are determined. (A).

Then a part of the lower jaw bone base D1 at a position adjacent to the predetermined cutting position of the cut-off piece of bone D2 is cut off, so as to form a flat cutoff surface 91 wherein the support pillar 10 is to be erected (B).

Then the prepared hole 92 for erecting the support pillar 10 is drilled in the cutoff surface 91 of the lower jaw bone base D1 (C).

Inner diameter of the prepared hole 92 is slightly smaller than that of the threaded portion 12 of the support pillar 10. Depth of the prepared hole 92 in the jaw bone base D1 is such that can firmly hold the support pillar 10 erected therein by screwing the threaded portion 12.

Then the support pillar 10 is screwed into the prepared hole 92, so as to erect the support pillar 10 in the lower jaw bone base D1 (D).

Then, although not shown, the mounting fixture 80 is fitted onto the support pillar 10 that has been erected and the mount base 82 of the mounting fixture 80 is brought into contact with the side face D of the bone to be cut off that adjoins the cutoff surface 91 on the contact surface 82C to make surface matching. Surface matching is an operation to cause the contact surface 82C of the fixing portion 82 to make sufficient contact with the side face D21 (refer to FIG. 8) of the bone to be cut off via the entire area thereof. After the surface matching, a screw hole (not shown) is drilled in the side face D21 of the bone to be cut off that corresponds to the set screw inserting hole 82a of the fixing portion 82.

After temporarily removing the support pillar 10 and the mounting fixture 80 from the prepared hole 92, the bone to be cut off is cut off along the predetermined line of cutoff P at the predetermined cutting position so as to realize the cut-off piece of bone D2 (E).

Then the support pillar 10 is screwed into the prepared hole 92 of the lower jaw bone base D1 thereby to erect therein (F).

With the mounting fixture 80 engaged with the adjust screw 20 by screwing in the screw receiving portion 81 thereof, the adjust screw 20 is fitted onto the support pillar 10, that has been erected, from the outside thereof, and the mount base 82 of the mounting fixture 80 is connected and fixed to the side face D21 of the cut-off piece of bone D2 (G).

The connecting fixation is carried out by screwing the set screw 83 through the set screw inserting hole 82c of the mounting fixture 82 into the screw hole that was drilled in the side face D2 in the previous step.

Then after suturing the mucous membrane E that was dissected, the living tissue protecting cap 30 is fitted onto the adjust screw 20 and the adjust screw 20 is turned so as to keep the lower jaw bone base D1 and the cut-off piece of bone D2 at a small distance from each other (H).

Thereafter, the adjust screw 20 is turned at predetermined intervals thereby to gradually increase the distance between the lower jaw bone base D1 and the cut-off piece of bone D2 gradually, so that the gap between the lower jaw bone base D1 and the cut-off piece of bone D2 is filled with newly generated bone, thereby extending the lower jaw bone D.

We claim:

1. A bone expanding apparatus that holds a bone base and a cut-off piece of bone, which is a part of the bone base being cut off therefrom, with a small separation gap and gradually increases said separation gap thereby to cause new bone to grow in said separation gap, thereby to fill said separation gap with the newly generated bone and expand the bone, comprising:
a support pillar to be erected and fixed in said bone base, an adjust screw that fits onto the support pillar from the outside thereof with said support pillar serving as a center axis of rotation and has thread formed on the circumference thereof, a screw receiving portion that receives the adjust screw being screwed therethrough, and a mounting fixture having mount base to be connected and fixed onto the cut-off piece of bone.

2. The bone expanding apparatus according to claim 1, wherein a stopper portion is provided at a mid position of the support pillar so as to make contact with and stop the distal end of the adjust screw.

3. The bone expanding apparatus according to claim 1, wherein the distal end portion of the support pillar is threaded so as to be screwed into the bone base by the threaded portion for fixing.

4. The bone expanding apparatus according to claim 3, wherein head of the support pillar has a joint at the head of the support pillar for detachably attaching a screw driver used as an auxiliary tool in screwing the threaded distal end portion into the bone base.

5. The bone expanding apparatus according to claim 1, wherein a living tissue protecting cap is detachably attached to the head of the adjust screw.

6. The bone expanding apparatus according to claim 1, wherein the support pillar that is erected and fixed in the bone base and the adjust screw that fits onto the support pillar from the outside thereof are erected while penetrating the cut-off piece of bone and a mount bases of the mounting fixture is mounted around the screw receiving portion through which said adjust screw penetrating therethrough by screwing.

7. The bone expanding apparatus according to claim 6, wherein an alignment assisting pin is provided as an auxiliary tool that is temporarily fitted in prepared holes drilled in the bone base and in the cut-off piece of bone prior to mounting of the support pillar and the adjust screw when connecting and fixing the mounting fixture onto the cut-off piece of bone, while the alignment assisting pin is fitted into the screw receiving portion of the mounting fixture at the same time, and has an outer diameter that just fits the prepared holes and an outer diameter that just fits the screw receiving portion of the mounting fixture.

8. The bone expanding apparatus according to claim 1, wherein the support pillar that is erected and fixed in the bone base and the adjust screw that fits onto the support pillar from the outside thereof are mounted at a position sideways to the cut-off piece of bone, and the mount bases of the mounting fixture is connected and fixed onto the cut-off piece of bone at a position sideways to the screw receiving portion through which the adjust screw penetrates by screwing.

9. The bone expanding apparatus according to claim 1, that is intended for use in expanding an alveolar bone or a jaw bone.

10. A bone expanding method of holding a bone base and a cut-off piece of bone, which is a part of the bone base being cut off therefrom, with a small separation gap from each other, and gradually increasing said separation gap thereby to cause new bone to grow in said separation gap, so that said separation gap is filled with the newly generated bone and expand the bone, the method comprising:
the step of drilling a prepared hole in the bone base for erecting the support pillar and drilling a prepared hole concentrically therewith in the cut-off piece of bone for passing the support pillar and the adjust screw therethrough, the step of temporarily fitting an alignment assisting pin in said prepared holes and fitting the screw receiving portion of the mounting fixture for fixing the cut-off piece of bone onto said alignment assisting pin thereby to connect and fix the mount bases of the mounting fixture into the cut-off piece of bone with the screw receiving portion of the mounting fixture being centered, the step of removing said alignment assisting pin from the prepared hole and passing the support pillar through the screw receiving portion of said mounting fixture and through the prepared hole of the cut-off piece of bone so as to erect and fix the support pillar in the prepared hole of the bone base, the step of fitting the adjust screw onto the support pillar, that has been erected, from the outside thereof with the support pillar serving as a center axis of rotation and screwing the thread formed on the circumference of the adjust screw into the internal thread of said screw receiving portion, and the step of turning the adjust screw so as to gradually expand the separation gap between the bone base and the cut-off piece of bone.

11. A bone expanding method of holding a bone base and a cut-off piece of bone, which is a part of the bone base being cut off therefrom, with a small separation gap from each other, and gradually increasing said separation gap thereby to cause new bone to grow in said separation gap, so that said separation gap is filled with the newly generated bone and expand the bone, the method comprising:
the step of forming a cut-off surface for erecting the support pillar by cutting into the bone at a position adjacent to a predetermined cutting position of the cut-off piece of bone, the step of forming a prepared hole in said cut-off surface for erecting the support pillar and temporarily erecting the support pillar therein, the step of fitting the mounting fixture for fixing the cut-off piece of bone onto the erected pin at the screw receiving portion thereof, putting the mount bases of the mounting fixture on the side face of the bone to be cut off that is adjacent to the cut-off surface for erecting the support pillar and drilling a mount hole in the bone to be cut off with the set screw inserting hole of the mounting fixture, the step of temporarily removing the support pillar and cutting off the cut-off piece of bone at said predetermined cutting position, the step of erecting the support pillar again in the bone base, the step of fitting the adjust screw onto the support pillar, that has been erected, from the outside thereof so as to turn around the support pillar serving as a center axis of rotation, the step of fixing the mount bases of the mounting fixture that has been screwed onto the thread formed on the circumference of the adjust screw into the cut-off piece of bone with the set screw, and the step of turning the adjust screw so as to gradually expand the separation gap between the bone base and the cut-off piece of bone.
